(19) United States
(12) Patent Application Publication
(10) Pub. No.: US 2003/0124239 A1
(43) Pub. Date: Jul. 3, 2003

(54) Water soluble animal muscle protein product

(76) Inventor: Stephen D. Kelleher, Wakefield, MA (US)

Correspondence Address: Paul J. Cook, 94 Central Street, Topsfield, MA, 01983, US

(21) Appl. No.: 10/367,026

(22) Filed: Feb. 19, 2003

Related U.S. Application Data
(63) Continuation-in-part of application No. 10/161,971, filed on Jun. 4, 2002.
Application No. 10/161,971 is a division of application No. 08/920,439, filed on Aug. 29, 1997, now Pat. No. 6,451,975.
Application No. 08/920,439 is a continuation-in-part of application No. 08/797,929, filed on Feb. 12, 1997, now Pat. No. 6,005,073.
(60) Provisional application No. 60/034,351, filed on Dec. 21, 1996.

(51) Int. Cl. A23L 1/238; A23L 1/40; C07K 14/47

(52) U.S. Cl. 426/589; 530/350

(57) ABSTRACT

A water soluble peptide composition derived from animal muscle tissue proteins is provided. The peptide composition contains less than about 1 weight percent fats and oils based upon the weight of the peptide composition and less than about 2 weight percent ash based on the weight of the peptide composition.

### REFERENCE TO RELATED APPLICATIONS

This application is a continuation-in-part application of application Ser. No. 10/161,971, filed Jun. 4, 2002, which, in turn, is a divisional application of application Ser. No. 08/920,439, filed Aug. 29, 1997, now U.S. Pat. No. 6,451,975 which, in turn, is a continuation-in-part of application Ser. No. 08/797,929, filed Feb. 12, 1997, now U.S. Pat. No. 6,005,073 which in turn is derived from Provisional application 60/034,351, filed Dec. 21, 1996.

### BACKGROUND OF THE INVENTION

This invention relates to a water soluble protein product and to a process for making the water soluble protein product. More particularly, this invention relates to such a water soluble product which is obtained from a protein derived from animal muscle tissue as a starting material in the process.

Prior to the present invention, animal proteins have been predigested with an enzyme in order to produce a peptone product, which can be utilized as a growth medium for microorganisms. Peptones are peptides utilized for bacterial growth. Unfortunately the composition of these peptone products can vary widely depending upon the source of the animal proteins so that the user has difficulty in effecting reproducible results. Components that can vary widely include fat and ash (mineral). In addition, these peptone products contain fats and oils, up to about 20 weight percent based on the peptone composition as well as ash, up to 12 weight percent based upon the weight of the peptone compositions. The fats, oils and ash undesirably do not contribute nutrients for a growth medium. Prior to the present invention, water soluble peptones or peptides derived from animal muscle tissue with low fat, low phosphorus, and low ash have not been available for human consumption.

Presently, protein derived from animal muscle for human consumption is obtained by processes wherein the protein is recovered at neutral or substantially neutral pH (pH 5.5-7.5), where a large fraction of the protein (myofibrillar) is fairly insoluble in water. Cortez-Ruiz et al, 2001 (J. Ag. Food Prod. Technol., 10(4):5-23) found proteins of bristly sardines extracted using acid solubilization to be only between 13-18% soluble in a high salt, when re-extracted using a neutral pH medium. Such processes are disclosed in U.S. Pat. Nos. 6,005,073; 6,288,216; 6,136,959 and 6,451,975. In addition this insoluble protein often has an undesirably dark brownish color, which renders it less desirable as a food additive. It is desirable to utilize food additives, which are white, substantially white or clear so that they do not substantially change the color of the food to which they are added.

It would be desirable to provide a form protein derived from animal muscle tissue which is soluble in water at neutral or substantially neutral pH and which retain their nutritional value. Such a form of protein can be utilized as a food grade additive for a wide variety of foods for human consumption including drinks, soups and solid foods. In addition, it would be desirable to provide such a form of protein derived from fish or meat, which is not less nutritional than the original form of the protein. Furthermore, it would be desirable to provide such a form of protein, which is low in fat, oil and ash which can be utilized to grow bacteria. Also, it would be desirable to provide such a form of food which is light in color such as white and which is clear when dissolved in water so that it does not change the color of food to which it can be added.

### SUMMARY OF THE INVENTION

In accordance with this invention, a mixture of myofibrillar proteins and sarcoplasmic proteins obtained by one of the processes disclosed in U.S. Pat. Nos. 6,005,073; 6,288,216 or 6,136,959 all of which are incorporated herein by reference in their entirety are digested with at least one enzyme to produce peptides which are soluble in water at neutral or substantially neutral pH; that is a pH between about 5.5 and about 7.5, preferably, between about 6.8 and about 7.1. The initial protein composition derived from animal muscle tissue comprises a mixture of myofibrillar proteins and sarcoplasmic proteins free of myofibrils and sarcomeres. The myofibrillar proteins are not soluble in water. Sarcoplasmic proteins, which are normally soluble in water, become fairly insoluble when in the presence of myofibrillar proteins that have spent any time at extreme pH (pH<3.5 or pH>10.5). The proteins can be in solid form or in acidic solution or alkaline solution when admixed with the enzyme composition. When the proteins are in acidic solution or alkaline solution, the pH of the solution can be adjusted to substantially neutral as set forth above after being digested with the enzyme composition. The enzyme composition can be active at acidic pH, alkaline pH or neutral pH. During enzyme digestion, the proteins are converted to water soluble peptides. Enzyme digestion can be stopped by changing the pH of the peptide solution to a pH where the enzyme composition is inactive. The reaction can also be stopped using heat. The enzyme composition can comprise one or more enzymes. The peptides can be recovered from solution by drying such as by spray drying, freeze drying or evaporation to obtain a dry peptide product. The dry peptide product is low in fat and oil primarily due to a centrifugation step in isolating the starting protein from fat and oil. The dry peptide product is low in ash primarily due to one or more washing steps of the protein to remove salt from the protein prior to digestion with the enzyme composition. In addition, it has been found that the enzyme digestion produces a dry peptide, which is lighter in color than the starting protein. This light color is important when the peptide is utilized for human consumption such as an additive to a conventional food product.

### DESCRIPTION OF SPECIFIC EMBODIMENTS

In accordance with this invention, a dry protein mixture or an aqueous acidic protein solution of myofibrillar proteins and sarcoplasmic proteins derived from animal muscle tissue and obtained by one of the processes disclosed in U.S. Pat. Nos. 6,005,073, 6,288,216 and 6,136,959 all of which are incorporated herein by reference in their entirety are utilized as a starting material in the process of this invention. The protein mixture is obtained by one of two processes. In one process, (acid process) animal muscle tissue is formed into small tissue particles which are then mixed with sufficient acid to form a solution of the tissue having a pH of 3.5 or less, but not such a low pH as to adversely modify the animal tissue protein. The solution is centrifuged to form a lowest membrane lipid layer, an intermediate layer of aqueous acidic protein solution and a top layer of neutral lipids (fats and oils). The intermediate layer of aqueous acidic protein solution then is separated from the membrane lipid layer or from both the membrane lipid layer and the neutral lipid layer. In this process, the protein mixture is free of myofibrils and sarcomeres. The protein in the aqueous acidic protein solution is recovered after centrifugation by drying the aqueous acidic solution, such as by evaporation, spray drying or lyophilization to form the dry protein mixture having the low pH it had when it was dissolved in the aqueous acidic protein solution. The dry protein mixture then is mixed with an enzyme composition in aqueous solution wherein the enzyme is active at acid pH. Alternatively, the aqueous acidic protein solution can be mixed with the acid-active enzyme composition without drying. It is preferred to utilize one of these two acid processes to obtain the dry protein mixture or the aqueous acidic protein solution that need not be dried prior to being admixed with the enzyme.

In a second process, (alkaline process) animal muscle tissue is formed into small tissue particles which are then mixed with sufficient aqueous base solution to form a solution of the tissue wherein at least 75% of the animal muscle protein is solubilized, but not such a high pH as to adversely modify the animal tissue protein. The solution is centrifuged to form a lowest membrane lipid layer, an intermediate aqueous protein rich layer and a top layer of neutral lipids (fats and oils). The intermediate aqueous protein-rich layer then is separated from the membrane lipid layer or from both the membrane lipid layer and the neutral lipid layer. The protein mixture is free of myofibrils and sarcomeres. The pH of the protein-rich aqueous phase then is lowered to a pH below about 3.5, preferably between about 2.0 and 3.5. The protein in the aqueous acidic solution is recovered after centrifugation by drying the aqueous acidic protein solution, such as by evaporation, spray drying or lyophilization to form a powder product having the low pH it had when it was dissolved in the aqueous acidic solution. Alternatively, the protein in aqueous acid solution is not dried prior to being mixed with an enzyme that is active at acid pH. The protein in aqueous basic solution having a pH above 8.5 and recovered after centrifugation is not dried, to form a powder product since these powders can be a source of health problems to a consumer in contrast to the dry composition recovered from the aqueous acidic solution discussed above. In one aspect of process, the pH of the basic solution can be lowered to about 5.5 to precipitate the protein. The pH of the precipitated protein then is raised to between 6.5 and 8.5 and a solid product is recovered such as by drying including spray drying, lyophilization or evaporation. The dry protein then is mixed with an enzyme composition in aqueous solution that is active at acidic, neutral or alkaline pH depending upon the pH of the protein product.

In summary, the dry protein mixture, the precipitated protein formed at pH 6.5-8.5 or the aqueous acidic protein solution utilized to produce the water soluble peptides of the present invention can be obtained by the following methods. It is preferred to utilize the protein starting composition derived from a process wherein the animal muscle tissue is dissolved in acid solution rather than in alkaline solution. This is due to the fact that animal protein dissolved in alkaline solution can form lysinoalanine, especially at elevated temperatures, which can cause renal disease in humans.

1. Reduce the pH of comminuted animal muscle tissue to a pH less than about 3.5 to form an acidic protein solution, centrifuge the solution to form a lipid-rich phase and an aqueous phase and recover an aqueous acidic protein solution substantially free of membrane lipids that can be used in this invention.

2. Spray dry the aqueous acidic protein solution obtained by method 1 to form a dry protein mixture substantially free of membrane lipids that can be used in the present invention.

3. Lyophilize the aqueous acidic protein solution obtained by method 1 to form the dry protein mixture substantially free of membrane lipids that can be used in the present invention.

4. Increase the pH of the aqueous acidic protein solution from method 1 to about pH 5.0-5.5 to effect precipitation of the proteins and then readjust the protein back to a pH of about 4.5 or less using acid in a minimum volume to concentrate the aqueous acidic protein solution to between 3.5-7% protein.

5. Increase the pH of comminuted animal muscle tissue to a pH above about 10.5, centrifuge the solution to form a lipid-rich phase and an aqueous phase and recover an aqueous basic protein solution. In one embodiment, reduce the pH of the aqueous basic solution to a pH of less than about 3.5 to obtain an aqueous acidic protein solution substantially free of membrane lipids that can be used in this invention. In a second embodiment, reduce the pH of the aqueous basic solution to about 5.0-5.5 to precipitate the protein, raise the pH of the precipitated protein to 6.5-8.5, dry and comminute the protein. In a third embodiment, reduce the pH of the aqueous basic solution to about 5.0-5.5 to precipitate the protein, lower the pH of the precipitated protein to a pH of 4.5 or less to form a concentrated aqueous acidic solution and use the concentrated aqueous acidic solution or dry the solution and use the recovered dry protein.

6. Spray dry the aqueous acidic protein solution obtained by method 5 to form a dry acidic protein mixture substantially free of membrane lipids that can be used in the present invention.

7. Lyophilize the aqueous acidic protein solution obtained by method 5 to form the dry acidic protein mixture substantially free of membrane lipids that can be used in the present invention.

8. Increase the pH of the aqueous, acidic protein solution from method 5 to about pH 5.0-5.5 to effect precipitation of the proteins and then readjust the protein back to a pH of about 4.5 or less using acid in a minimum volume to concentrate the aqueous acidic solution to between 3.5-7% protein.

The protein products utilized in the present invention comprise primarily myofibrillar proteins that also contain significant amounts of sarcoplasmic proteins. The sarcoplasmic proteins in the protein starting composition which is admixed with the enzyme comprises above about 8%, preferably above about 10%, more preferably above about 15% and most preferably above about 18%, up to about 30% by weight sarcoplasmic proteins, based on the total weight of protein in the dry acidic protein mixture, the precipitated protein formed at pH 6.5-8.5 or aqueous acidic protein solution.

The starting protein is derived from meat or fish, including shellfish. Representative suitable fish include deboned flounder, sole haddock, cod, sea bass, salmon, tuna, trout or the like. Representative suitable shellfish include shelled shrimp, crayfish, lobster, scallops, oysters or shrimp in the shell or the like. Representative suitable meats include beef, lamb, pork, venison, veal, buffalo or the like; poultry such as chicken, mechanically deboned poultry meat, turkey, duck, a game bird or goose or the like.

In accordance with this invention the dry protein mixture or aqueous solution of myofibrillar proteins and sarcoplasmic proteins is mixed with one or more enzymes, which convert the protein to peptides. The enzymes can be exoproteases or endoproteases and can be active to produce peptides at an acidic pH, an alkaline pH or a neutral pH. Representative suitable enzymes useful at acidic pH include Enzeco Fungal Acid Protease (Enzyme Development Corp., New York, N.Y.; Newlase A (Amano, Troy, Va.); and Milezyme 3.5 (Miles Laboratories, Elkhart, Ind.) or mixtures thereof. Representative suitable enzymes useful at alkaline pH include Alcalase 2.4 LFG (Novozymes, Denmark). Representative suitable enzymes useful at neutral pH include Neutrase 0.8L (Novozymes, Denmark) and papain (Penta, Livingston, N.J.) or mixtures thereof.

The enzymes are utilized in amounts of between about 0.02% and about 2% preferably between about 0.05% and about 0.5% by weight based on the total weight of enzyme and protein at temperatures between about 40° C. and about 55° C. preferably between about 25° C. and about 40° C., for a time between about 5 mins. and about 24 hrs., preferably between about 0.5 hrs. and about 2 hrs.. The peptides formed by reaction of the protein composition with the enzyme composition then are recovered by drying the solution wherein the reaction takes place. Drying can be effected by evaporation, spray drying, freeze-drying or the like. The peptides produced by the present invention are instantaneously soluble in water at neutral pH.

The peptide products of this invention contain less than about 1 weight percent fats and oils (total), preferably less than about 0.2% weight percent fats and oils based on the weight of peptide. In addition, the peptide products of this invention contain less than about 2 weight percent ash, preferably less than about 0.9% weight percent ash based on the weight of peptide. This low ash content is achieved by washing with water the protein starting material. Ash is defined as minerals, such as sodium, potassium, calcium, iron or phosphorus. In addition, the peptide products of this invention are instantly soluble in water to form a clear solution. Furthermore, the peptide products of this invention generally have lighter color whiteness units than the color whiteness units of a similar unhydrolyzed protein isolate from which they are derived as measured by a colorimeter with L,a,b capabilities. This lighter color is found with the hydrolyzed peptides of this invention derived from meats such as beef, pork or chicken as well as from dark muscle tissue from fish such as pelagic fish as shown for example in Example 1 below. This lighter color characteristic is desirable since it is more easily permits dissolving the peptide product in water to form clear aqueous solutions.

Color whiteness index is determined by converting the L,a,b values utilizing the formula: 100[(100−L)²+a²+b²]^{0.5}. Color is measured using a tristimulus calorimeter utilizing the universally adopted “L, a, b” opponent-type scale developed by Richard Hunter as is well known in the art. “L” is a measure of light ranging from white to black. The “a” value measures the range from green to red, and the “b” value measures the range from blue to yellow. With these three coordinates, a three-dimensional value can be assigned to any color.

In one aspect of this invention, a growth medium for microorganisms is provided which is in gel form and which contains the peptide products of this invention in a concentration, which provides growth nutrients to the microorganism being grown. The peptide products comprise between about 0.5 and about 10 percent, preferably between about 1 percent and about 5 percent by weight of the peptide based on the total weight of the peptide and the gel component of the growth medium. At peptide concentrations above about 10 weight percent, difficulties are encountered in forming and maintaining the gel structure. The gel component of the growth medium comprises the dry protein mixture, the precipitated protein formed at pH 6.5 -8.5 or the aqueous acidic protein solution starting materials utilized to produce the water soluble peptides of the present invention. The gel is formed from these starting materials by placing the protein into a mini chopper that is pre-chilled with ice. Two (2%) percent aqueous NaCl is added to the chopper and the material is chopped between 2-3 min. The protein paste is placed into a polymeric, e.g. polyethylene bag and all the air is removed by hand pressing. The paste is rolled to a thickness of 3 mm and placed for 25 seconds on high in a microwave oven, and then cooled. The final cooled material is tested for its ability to double-fold and rated on a 5 point test as described by Kudo et al. (1973, Marine Fish. Rev. 32:10-15). The mixture of the peptide and gel component can be formed by partially reacting the gel component with an enzyme as described above or by hydrolyzing the protein starting material as described above and then mixing the gel with the hydrolyzed product. The gel-peptide composition can be utilized as a growth medium for microorganism on the surface of the gel-peptide composition under temperature conditions that promotes microorganism growth as is well-known in the art. The gel-peptide mixture also can be added to a food for human consumption to provide nutrients for a human consumer of the food.

The following examples illustrate the present invention and are not intended to limit the same.

### EXAMPLE 1

Hydrolysis at Acidic pH

Chicken protein isolate from myofibrillar and sarcoplasmic proteins was produced according to U.S. Pat. No. 6,005,073 (pH 2.8; 10,000 g-force) from fresh chicken breast muscle and adjusted to pH 5.5 to precipitate the proteins. The precipitated proteins were then readjusted back to pH 3.5 using hydrochloric acid (2 N) added drop-wise. No additional liquid other than the acid was added. Protein concentration of the acidified protein was 49.86 mg/ml. Two aliquots of the protein sample were placed into glass beakers and placed into a water bath at 50° C. In one of the beakers 0.05% (w/w) of S-16774 Enzeco Fungal Acid Protease (Enzyme Development Corporation, 21 Penn Plaza, 360 West 31^{st} St., New York, N.Y.) was mixed by spatula to disperse. The samples were incubated at 50° C. for 2.3 hours. Both samples were subsequently adjusted to pH 7.0 using sodium hydroxide (2N) added drop-wise. The sample with no added enzyme precipitated at pH 5.5, but then returned to a liquid appearance upon reaching the neutral pH. The sample with added enzyme remained a liquid throughout the pH 3.5 to 7.0 range. Both samples were then placed into a refrigerator. Portions of both samples were freeze-dried until approximately 5% moisture and stored in Whirl-Pak bags.

Sample Characteristics:

| Characteristic | Enzyme added | No enzyme added |
|---|---|---|
| Viscosity (cps.)¹ | 3.9 | 46.2 |
| Color² | Light cream | Tan |
| Odor² | v. slight chicken | mild chicken |
| Solubility (%)³ | 97.3 | 15.8 |
| Whiteness Index^{2,4} | 71.85 | 62.78 |
| Rehydrate/Dispersability² | Almost immediate | v. poor |
| ¹Determined using a Brookfield HAT viscometer, 100 RPM, Spindle #5 | | |
| ²Samples were evaluated as freeze-dried powders. | | |
| ³Solubility was determined by homogenizing (speed 1 for 30 secs., PowerGen 700, Fisher Scientific) 1 part protein to 9 parts 100 mM sodium phosphate (monobasic, anhydrous) buffer, pH 7, prior to centrifuging at 5,000 g-force for 20 min using an Eppendorf Mini-spin. Protein content was determined using the biuret method of Torten, J. and Whitaker, J. R. (1969, J Food Sci. 29: 168-174) on the homogenate and the supernate fractions. Solubility was expressed as gram | | |
| # protein in supernate/gram protein homogenate, times 100. | | |

⁴Whiteness Index was obtained using 100 − [(100 − L)² + a² + b²]^{0.5}. L, a, b values obtained using a Minolta CR-10 color meter.

Analysis of Freeze-Dried, Hydrolyzed Chicken Proteins

| Component | Amount (%) |
|---|---|
| Protein | 94.3 |
| Fat | 0.2 |
| Ash | 0.9 |
| Carbohydrate | 0.2 |
| Moisture | 4.4 |

Methods A.O.A.C. 15^{th} Ed., 1995.

### EXAMPLE 2

Hydrolysis at Neutral pH

Chicken protein isolate from myofibrillar and sarcoplasmic proteins was produced according to U.S. Pat. No. 6,005,073 (pH 2.8; 10,000 g-force) from fresh chicken breast muscle and adjusted to pH 5.5 to precipitate the proteins. The precipitated proteins were then adjusted to pH 7 using sodium hydroxide (2 N) added drop-wise. No additional liquid other than the acid was added. Two aliquots of the protein sample were placed into glass beakers and placed into a water bath at 40° C. In one of the beakers 0.2% (w/w) of Neutrase 0.8 L (Batch PWNO1208; Novozymes A/S, Krogshoejvej 36, 2880 Bagsvaerd, Denmark) was mixed by spatula to disperse. The samples were incubated at 40° C. for 0.5 hours. Both samples were then placed into a refrigerator prior to measuring viscosity and solubility.

Sample Characteristics:

| Characteristic | Enzyme added | No enzyme added |
|---|---|---|
| Viscosity (cps.)¹ | 9.4 | 65.9 |
| Solubility (%)² | 100 | 13.3 |
| ¹Brookfield HAT viscometer, 100 RPM, Spindle #5 | | |
| ²Solubility was determined by homogenizing (speed 1 for 30 secs., PowerGen 700, Fisher Scientific) 1 part protein to 9 parts 100 mM sodium phosphate (monobasic, anhydrous) buffer, pH 7, prior to centrifuging at 5,000 g-force for 20 min using an Eppendorf Mini-spin. Protein content was determined using the biuret method of Torten, J. and Whitaker, J. R. (1969, J Food Sci. 29: 168-174) on the homogenate and the supernate fractions. Solubility was expressed as gram | | |
| # protein in supernate/gram protein homogenate, times 100. | | |

### EXAMPLE 3

Hydrolysis at Alkaline pH

Chicken protein isolate from myofibrillar and sarcoplasmic proteins was produced according to U.S. Pat. No. 6,005,073 (pH 2.8; 10,000 g-force) from fresh chicken breast muscle and adjusted to pH 5.5 to precipitate the proteins. The precipitated proteins were then adjusted to pH 8.0 using sodium hydroxide (2 N) added drop-wise. No additional liquid other than the acid was added. Two aliquots of the protein sample were placed into glass beakers and placed into a water bath at 55° C. In one of the beakers 0.5% (w/w) of Alcalase 2.4 L FG, (Batch PLN05212; Novozymes A/S, Krogshoejvej 36, 2880 Bagsvaerd, Denmark) was mixed by spatula to disperse. The samples were incubated at 55° C. for 1.5 hours. Both samples were then adjusted to pH 7 and placed into a refrigerator prior to measuring viscosity and solubility.

Sample Characteristics:

| Characteristic | Enzyme added | No enzyme added |
|---|---|---|
| Viscosity (cps.)¹ | 1.9 | 72.8 |
| Solubility (%)² | 99.4 | 26.2 |
| ¹Brookfield HAT viscometer, 100 RPM, Spindle #5 | | |
| ²Solubility was determined by homogenizing (speed 1 for 30 secs., PowerGen 700, Fisher Scientific) 1 part protein to 9 parts 100 mM sodium phosphate (monobasic, anhydrous) buffer, pH 7, prior to centrifuging at 5,000 g-force for 20 min using an Eppendorf Mini-spin. Protein content was determined using the biuret method of Torten, J. and Whitaker, J. R. (1969, J Food Sci. 29: 168-174) on the homogenate and the supernate fractions. Solubility was expressed as gram | | |
| # protein in supernate/gram protein homogenate, times 100. | | |

### EXAMPLE 4

Gel Made from a Mixture of Unhydrolyzed and Hydrolyzed Protein Isolates

Methods

Chicken protein isolate, from myofibrillar and sarcoplasmic proteins, was produced according to U.S. Pat. No. 6,005,073 (pH 2.8; 10,000 g-force) from fresh chicken breast muscle and adjusted to pH 5.5 to precipitate the proteins. The precipitated proteins were then readjusted back to pH 3.5 using hydrochloric acid (2 N) added drop-wise. No additional liquid other than the acid was added. Protein concentration of the acidified protein was 49.86 mg/ml. The protein sample was placed into glass beakers and placed into a water bath at 50° C. In a beaker 0.05% (w/w) of S-16774 Enzeco Fungal Acid Protease (Enzyme Development Corporation, 21 Penn Plaza, 360 West 31^{st} St., New York, N.Y.) was mixed by spatula to disperse. The sample was incubated at 50° C. for 2.3 hours. The sample was subsequently adjusted to pH 7.0 using sodium hydroxide (2N) added drop-wise. The sample with added enzyme remained a liquid throughout the pH 3.5 to 7.0 range. The sample was placed into a refrigerator. The sample was freeze-dried until approximately 5% moisture and stored in Whirl-Pak bags (“HYDROLYZED”).

Pork protein isolate from myofibrillar and sarcoplasmic proteins was produced according to U.S. Pat. No. 6,005,073 (pH 2.8; 10,000 g-force) from fresh pork muscle and adjusted to pH 5.5 to precipitate the proteins. The precipitate was adjusted to pH 7.0 and freeze-dried until approximately 5% moisture and stored in Whirl-Pak bags (“UNHYDROLYZED”).

A gel was manufactured using “HYDROLYZED” chicken protein and “UNHYDROLYZED” pork protein isolate as follows: “UNHYDROLYZED” pork powder (20.01 g), was added to a Procter-Silex mini chopper along with 74.84 g of an ice/cold water mixture, 0.98 g NaCl, and 0.94 g of “HYDROLYZED” chicken protein. The mixture was blended for approximately 4 min or until a final temperature of 8° C. was reached. The protein paste was placed into a Whirl-Pak bag and all the air was removed by hand pressing. The paste was rolled to a thickness of 3 mm and placed for 25 s on high in a Sharp Carousel microwave oven, and then cooled. The final cooled material was tested for its ability to double-fold and rated on a 5 point test as described by Kudo et al. (1973, Marine Fish. Rev. 32:10-15). A sample with no breaks after being double folded was rated the highest at 5.

Results

Six samples out of six of the protein isolate mixture of “HYDROLYZED” and “UNHYDROLYZED” gels were found to rate a score of 5, with no detectable breaks upon being double-folded. The material was a pleasant tan, brown color, with a slight cooked pork odor.

### EXAMPLE 5

Gelation and Solubility of Cod Muscle Proteins Partially Hydrolyzed Using Acid Stable Enzyme.

Methods

Cod protein isolate from myofibrillar and sarcoplasmic proteins was produced according to U.S. Pat. No. 6,005,073 (pH 2.8; 10,000 g-force) from fresh cod muscle. One aliquot of the solubilized muscle proteins were placed into a large Whirl-Pak bag to which enzyme was added. In the bag 0.05% (w/w) of S-16774 Enzeco Fungal Acid Protease (Enzyme Development Corporation, 21 Penn Plaza, 360 West 31^{st} St., New York, N.Y.) was mixed to disperse (mixing was done by hand to simulate a Stomacher mixer). The sample was incubated at 45° C. (pH 2.8) for 20 min. The sample was subsequently adjusted to pH 5.5 using sodium hydroxide (2N) added drop-wise to precipitate the proteins. The precipitate was de-watered using a centrifugal force of 11,000×g in a Sorvall RC-5B centrifuge. Additional moisture was hand squeezed out of the protein until dry to touch. The precipitated proteins were then adjusted to pH 7 using sodium hydroxide (2N) added drop-wise. No additional liquid other than the acid was added. The de-watered protein isolate was mixed with 5% sorbitol, 4% sucrose and 0.3% sodium tripolyphosphate and frozen (−30° C.) in Whirl-Pak bags.

Gels were made by thawing the samples at room temperature until breakable, but not soft. The protein was placed into a Proctor-Silex mini chopper that was pre-chilled with ice. Two (2%) percent NaCl was added to the chopper and the material was chopped between 2-3 min. The protein paste was placed into a Whirl-Pak bag and all the air was removed by hand pressing. The paste was rolled to a thickness of 3 mm and placed for 25 s on high in a Sharp Carousel microwave oven, and then cooled. The final cooled material was tested for its ability to double-fold and rated on a 5 point test as described by Kudo et al. (1973, Marine Fish. Rev. 32:10-15). A sample with no breaks after being double folded was rated the highest at 5. Protein content was determined using the biuret method of Torten, J. and Whitaker, J. R. (1969, J Food Sci. 29:168-174) on the homogenate and the supernate fractions. Protein solubility was expressed as gram protein in supernate/gram protein homogenate, times 100.

The control underwent all the above steps minus the 20 min incubation step with the enzyme.

Results

Both the control and the enzyme incubated samples were found to score a 5 on the double-fold test. A score of 5 describes a gel with no visible cracks found after the material was double-folded. Protein solubility on the control was found to be 17.3% ±3.6. Solubility on the enzymatically treated sample was 31.2±2.9.

1. A water soluble peptide composition derived from animal muscle tissue proteins, consisting of myofibrils and sarcoplasmic fractions, said peptide composition containing less than about 1 weight percent fats and oils based upon the weight of the peptide composition and less than about 2 weight percent ash based on the weight of the peptide composition.

2. The peptide composition of claim 1 containing less than about 0.2 weight percent fats and oils based upon the weight of the peptide composition and less than about 0.9 weight percent ash based upon the weight of the peptide composition.

3. The composition of any one of claims 1 or 2 wherein said animal muscle tissue is fish.

4. The composition of any one of claims 1 or 2 wherein said animal muscle tissue is shellfish.

5. The composition of claim 4 wherein said shellfish is shrimp.

6. The composition of any one of claims 1 or 2 wherein said animal muscle tissue is meat.

7. The composition of any one of claims 1 or 2 wherein said animal muscle tissue is poultry.

8. The composition of claim 7 wherein said animal muscle tissue is selected from the group consisting of duck, turkey, goose, game bird and chicken.

9. The composition of claim 6 wherein said animal muscle tissue is selected from the group consisting of beef, lamb, pork, veal, buffalo and venison.

10. The process for forming a water soluble peptide composition from a protein composition derived from animal muscle tissue, which comprises:
(a) providing a protein mixture selected from the group consisting of a dry protein mixture of myofibrillar proteins and sarcoplasmic proteins derived from animal muscle tissue, an aqueous acidic protein solution of myofibrillar proteins and sarcoplasmic proteins derived from animal muscle tissue and mixtures thereof
(b) mixing said protein mixture from step (a) with an enzyme composition which forms said peptide composition from said protein mixture and
(c) recovering said peptide composition.

11. The process of claim 10 wherein said protein mixture is a dry protein mixture formed by drying an acidic aqueous solution of said protein mixture.

12. The process of any one of claims 10, or 11 wherein said animal muscle tissue is fish.

13. The process of any one of claims 10 or 11 wherein said animal muscle tissue is shellfish.

14. The process of claim 13 wherein said shellfish is shrimp.

15. The process of claims 10 or 11 wherein said animal muscle tissue is poultry.

16. The process of claim 15 wherein said animal muscle tissue is selected from the group consisting of turkey, duck, goose, game bird and chicken.

17. The process of any one of claims 10 or 11 wherein said animal muscle tissue is meat.

18. The process of claim 17 wherein said animal muscle tissue is selected from the group consisting of ham, beef, lamb, pork, veal, buffalo and venison.

19. A gel composition comprising a gel derived from a protein mixture selected from the group consisting of a dry protein mixture of myofibrillar proteins and sarcoplasmic proteins derived from animal muscle tissue, an aqueous acidic protein solution of myofibrillar proteins and sarcoplasmic proteins derived from animal muscle tissue and mixtures thereof and between about 0.5 and about 10 percent by weight of the peptide of claim 1 based on the total weight of the peptide and the gel component of the growth medium.

20. A gel composition comprising a gel derived from a protein mixture selected from the group consisting of a dry protein mixture of myofibrillar proteins and sarcoplasmic proteins derived from animal muscle tissue, an aqueous acidic protein solution of myofibrillar proteins and sarcoplasmic proteins derived from animal muscle tissue and mixtures thereof and between about 1 and about 5 percent by weight of the peptide of claim 1 based on the total weight of the peptide and the gel component of the growth medium.

21. A gel composition comprising a gel derived from a protein mixture selected from the group consisting of a dry protein mixture of myofibrillar proteins and sarcoplasmic proteins derived from animal muscle tissue, an aqueous acidic protein solution of myofibrillar proteins and sarcoplasmic proteins derived from animal muscle tissue and mixtures thereof and between about 0.5 and about 10 percent by weight of the peptide of claim 2 based on the total weight of the peptide and the gel component of the growth medium.

22. A gel composition comprising a gel derived from a protein mixture selected from the group consisting of a dry protein mixture of myofibrillar proteins and sarcoplasmic proteins derived from animal muscle tissue, an aqueous acidic protein solution of myofibrillar proteins and sarcoplasmic proteins derived from animal muscle tissue and mixtures thereof and between about 1 and about 5 percent by weight of the peptide of claim 2 based on the total weight of the peptide and the gel component of the growth medium.
